(19) United States
(12) Patent Application Publication
(10) Pub. No.: US 2011/0275606 A1
(43) Pub. Date: Nov. 10, 2011

(54) SUBSTITUTED PORPHYRINS

(75) Inventors: Ines Batinic-Haberle, Durham, NC (US); Julio S. Reboucas, Joao Pessoa (BR); Ivan Spasojevic, Durham, NC (US)

(73) Assignee: DUKE UNIVERSITY, Durham, NC (US)

(21) Appl. No.: 13/143,672

(22) PCT Filed: Jan. 7, 2010
(86) PCT No.: PCT/US10/20328

§ 371 Date: Jul. 7, 2011

Related U.S. Application Data
(60) Provisional application No. 61/142,970, filed on Jan. 7, 2009.

(51) Int. Cl. A61K 31/555; C12N 5/07; A61P 25/00; A61P 35/00; A61P 29/00; A61P 9/00; A61P 19/02; A61P 37/06; A61P 3/10; A61P 25/30; C07F 15/00; A61P 9/10

(52) U.S. Cl. 514/185; 546/2; 435/375

(57) ABSTRACT

Discloses are compounds according to Formula I. Further provided are methods of reducing oxidative stress in a cell, methods of treating a disease including cancer, and methods of treating a subject with a disorder associated with oxidative stress, the methods including administering a compound according to the invention. Also disclosed are methods of potentiating a cancer cell for treatment with ionizing radiation or chemotherapeutics including contacting the cell with an effective amount of a compound according to the invention. Further discloses are pharmaceutical compositions comprising a pharmaceutically acceptable carrier and a compound according to the invention.

### CROSS-REFERENCE TO RELATED APPLICATIONS

This application claims priority to U.S. Provisional Application No. 61/142,970, filed Jan. 7, 2009, incorporated by reference in its entirety.

### STATEMENT OF GOVERNMENT SUPPORT

This invention was made with US Government support awarded by National Institutes of Health, Grant No. U19A167798-01. The United States has certain rights in this invention.

### SUMMARY OF THE INVENTION

Provided are compounds according to Formula I, as detailed below.

Further provided are methods of reducing oxidative stress in a cell, the method including contacting the cell with an effective amount of a compound according to Formula I, as detailed below.

Further provided are methods of treating a disease or disorder, the method including administering to a patient in need thereof an effective amount of a compound according to Formula I, as detailed below. The disease or disorder may be selected from the group consisting of central nervous system injuries, stroke, spinal cord injury, cancer, ischemia/reperfusion injuries, cardiovascular injuries, arthritis, sickle cell disease, radiation injury, auto-immune diseases, diabetes, morphine tolerance, drug dependence/addiction and inflammatory conditions.

Further provided are methods of treating cancer, the method including administering to a patient in need thereof an effective amount of a compound according to Formula I, as detailed below. The cancer may be selected from the group consisting of lung, breast, brain, skin, head and neck, prostate, pancreas, gastrointestinal, and colon cancer.

Also provided are pharmaceutical compositions comprising a pharmaceutically acceptable carrier and a compound according to Formula I, as detailed below.

Further provided are methods of treating a subject with a disorder associated with oxidative stress, the method including administering to the subject an effective amount of a compound according to Formula I, as detailed below.

Further provided are methods of potentiating a cancer cell for treatment with ionizing radiation or chemotherapeutics, the method including contacting the cancer cell with an effective amount of a compound according to Formula I, as detailed below.

### BRIEF DESCRIPTION OF THE DRAWINGS

FIG. 1 shows schemes for the chemical synthesis of substituted porphyrins according to some embodiments of the present invention.

FIG. 2 is a graph of absorbance at 700 nm, showing the aerobic growth of wild type (GC) and SOD-deficient strains (QC) of E. coli in minimal medium in the presence and absence of MnTE-2-PyP⁵⁺ and its alcohol analogue MnTnPrOH-3-PyP⁵⁺.

FIG. 3 is a graph of absorbance at 700 nm, showing the aerobic growth of wild type (AB) and SOD-deficient strains (JI) of E. coli in minimal medium in the presence and absence of MnTE-2-PyP⁵⁺ and its alcohol analogue MnTnPrOH-3-PyP⁵⁺.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

A new class of substituted porphyrins has been discovered. The substituted porphyrins according to the present invention can be used to reduce or modulate oxidative stress and to treat disease conditions resulting, at least in part, from oxidative stress injury. The substituted porphyrins may also be used as an adjuvant in radiation therapy or chemotherapy or pain management, such as reducing morphine tolerance. The substituted porphyrins of the present invention may be administered in combination with other active agents, such as anti-cancer agents, anti-inflammatory agents, and analgesics. The substituted porphyrins may increase the bioavailability of other active agents. In addition, the substituted porphyrins according to the present invention may act synergistically in combination with other active agents. The invention will be described in greater detail below.

### DEFINITIONS

“Acyl” or “carbonyl” refers to the group —C(O)R wherein R is alkyl, alkenyl, alkynyl, aryl, heteroaryl, carbocyclic, heterocarbocyclic, C₁₋₄ alkyl aryl or C₁₋₄ alkyl heteroaryl. C₁₋₄ alkylcarbonyl refers to a group wherein the carbonyl moiety is preceded by an alkyl chain of 1-4 carbon atoms.

“Alkenyl” refers to an unsaturated aliphatic hydrocarbon moiety including straight chain and branched chain groups. Alkenyl moieties must contain at least one double bond. Suitably, an alkenyl moiety has from 2 to 10 carbon atoms. In some embodiments, the alkenyl has no more than 8 carbons or no more than 5 carbons or at least 3 carbons. “Alkenyl” may be exemplified by groups such as ethenyl, n-propenyl, isopropenyl, n-butenyl and the like. Alkenyl groups may be substituted or unsubstituted or branched or unbranched. More than one substituent may be present. Substituents may also be themselves substituted. Substituents can be placed on the alkene itself and also on the adjacent member atoms or the alkenyl moiety. “C₂₋₄ alkenyl” refers to alkenyl groups containing two to four carbon atoms.

“Alkoxy” refers to the group —O—R wherein R is acyl, alkyl alkenyl, alkyl alkynyl, aryl, carbocyclic, heterocarbocyclic, heteroaryl, C₁₋₄ alkyl aryl or C₁₋₄ alkyl heteroaryl. For example, methoxy or ethoxy.

“Alkyl” refers to a monovalent alkyl group, such as methyl, ethyl, propyl, etc. In some embodiments, the alkyl has from 1 to 10 carbon atoms. In other embodiments, the alkyl has no more than 8 carbon atoms or no more than 6 carbon atoms. In other embodiments, the alkyl group has at least 3 carbon atoms. The alkyl group can be saturated or unsaturated, branched or unbranched, and substituted or unsubstituted. Substituents may also be substituted.

“Alkylene” refers to a divalent alkyl group, such as methylene (—CH₂—), ethylene (—CH₂—CH₂—), propylene (—CH₂—CH₂—CH₂—), hexylene (—CH₂CH₂CH₂CH₂CH₂CH₂—) etc. In some embodiments, the alkylene has from 1 to 10 carbon atoms. In other embodiments, the alkylene has no more than 8 carbon atoms or no more than 6 carbon atoms. In further embodiments, the alkylene group has at least 3 carbon atoms. In some embodiments, the alkylene group has from 3 to 6 carbon atoms. In some embodiments, the alkylene has from 6 to 10 carbon atoms. In others, the alkylene has from 4 to 10 carbon atoms. In some embodiments, one or more of the carbon atoms is replaced by a heteroatom. The alkylene group may be saturated or unsaturated. The alkylene group may suitably be branched and in some embodiments, the branched alkylene group forms a carbocycle or aryl group. In addition, the alkylene group may be substituted. An unsubstituted alkylene may be written as —(CH₂)ₙ—, where n can be from 1 to 10 (as discussed above).

“Alkynyl” refers to an unsaturated aliphatic hydrocarbon moiety including straight chain and branched chain groups. Alkynyl moieties must contain at least one triple bond. Alkynyl moieties suitably have from 2 to 10 carbons. In some embodiments, the alkynyl has no more than 8 carbons or no more than 5 carbons or at least 3 carbons. “Alkynyl” may be exemplified by groups such as ethynyl, propynyl, n-butynyl and the like. Alkynyl groups may be substituted or unsubstituted or branched or unbranched. More than one substituent may be present. Substituents may also be themselves substituted. Substituents are not on the alkyne itself but on the adjacent member atoms of the alkynyl moiety. “C₂₋₄ alkynyl” refers to alkynyl groups containing two to four carbon atoms.

“Amino” refers to the group —NR′R′ wherein each R′ is, independently, hydrogen, amino, hydroxyl, alkoxyl, alkyl, aryl, cycloalkyl, heterocycloalkyl, heteroaryl, C₁₋₄ alkyl aryl or C1-4 alkyl heteroaryl. The two R′ groups may themselves be linked to form a ring. The R′ groups may themselves be further substituted.

“Aryl” refers to an aromatic carbocyclic group. Suitably, aryl has 5 to 10 carbons and may be monocyclic or bicyclic. In some embodiments, the aryl group has 5 to 6 carbons and in other embodiments, the aryl group may have 9 to 10 carbons. “Aryl” may be exemplified by phenyl or naphthalene or cyclopentadienyl. The aryl group may be substituted or unsubstituted. More than one substituent may be present. Substituents may also be themselves substituted. When substituted, the substituent group is preferably but not limited to heteroaryl, acyl, carboxyl, carbonylamino, nitro, amino, cyano, halogen, or hydroxyl.

“Carboxyl” refers to the group —C(═O)O—R, wherein each R is, independently, hydrogen, alkyl, aryl, cycloalkyl, heterocycloalkyl, heteroaryl, C₁₋₄ alkyl aryl or C₁₋₄ alkyl heteroaryl.

“Carbonyl” refers to the group —C(O)R wherein each R is, independently, hydrogen, alkyl, aryl, cycloalkyl, heterocycloalkyl, heteroaryl, C₁₋₄ alkyl aryl or C₁₋₄ alkyl heteroaryl.

“Carbonylamino” refers to the group —C(O)NR′R′ wherein each R′ is, independently, hydrogen, alkyl, aryl, cycloalkyl, heterocycloalkyl, heteroaryl, C₁₋₄ alkyl aryl or C₁₋₄ alkyl heteroaryl. The two R′ groups may themselves be linked to form a ring.

“C₁₋₄ alkyl aryl” refers to C₁₋₄ alkyl groups having an aryl substituent such that the aryl substituent is bonded through an alkyl group. “C₁₋₄ alkyl aryl” may be exemplified by benzyl.

“C₁₋₄ alkyl heteroaryl” refers to C₁₋₄ alkyl groups having a heteroaryl substituent such that the heteroaryl substituent is bonded through an alkyl group.

“Carbocyclic group” or “cycloalkyl” means a monovalent saturated or unsaturated hydrocarbon ring. Carbocyclic groups are monocyclic, or are fused, spiro, or bridged bicyclic ring systems. Monocyclic carbocyclic groups contain 3 to 10 carbon atoms, suitably 4 to 7 carbon atoms, or 5 to 6 carbon atoms in the ring. Bicyclic carbocyclic groups contain 8 to 12 carbon atoms, suitably 9 to 10 carbon atoms in the ring. Carbocyclic groups may be substituted or unsubstituted. More than one substituent may be present. Substituents may also be themselves substituted. Suitable carbocyclic groups include cyclopropyl, cyclobutyl, cyclopentyl, cyclohexyl, cyclohexenyl, and cycloheptyl. Carbocyclic groups are not aromatic.

“Disulfide” refers to the group —S—S—R, wherein R is alkyl, aryl, heteroaryl, C₁₋₄ alkyl aryl or C₁₋₄ alkyl heteroaryl.

“Effective amount” refers to a dosage of the compounds or compositions effective for eliciting a desired effect.

“Excipient” as used herein includes physiologically compatible additives useful in preparation of a pharmaceutical composition. Examples of pharmaceutically acceptable carriers and excipients can for example be found in Remington Pharmaceutical Science, 16th Ed.

“Halogen” refers to a fluoro, chloro, iodo or bromo.

“Heteroalkyl” refers to an alkyl group containing one or more heteroatoms.

“Heteroaryl” refers to a 5 or 10 membered aromatic ring which contains 1 or more heteroatoms. Suitably, the heteroaryl group has 5 to 6 members or 9 to 10 members. If more than one heteroatom is present, the heteroatoms may be the same or different. The heteroaryl groups are optionally substituted. In some embodiments, the heteroaryl group has a nitrogen at the ortho position. In some embodiments, the heteroaryl group has a nitrogen at the meta position. Suitably, the heteroaryl group has 1 nitrogen, 2 nitrogens or 3 nitrogens, such as pyridyl, imidazolyl, pyrazolyl, pyrimidyl and thiazolyl.

“Heteroatom” refers to a nitrogen, sulfur or oxygen. The heteroatom may be substituted in some embodiments. Groups containing more than one heteroatom may contain different heteroatoms.

“Heterocarbocyclic group” or “heterocycloalkyl” or “heterocyclic” means a monovalent saturated or unsaturated hydrocarbon ring containing at least one heteroatom. Heterocarbocyclic groups are monocyclic, or are fused, spiro, or bridged bicyclic ring systems. Monocyclic heterocarbocyclic groups contain 3 to 10 carbon atoms, suitably 4 to 7 carbon atoms, or 5 to 6 carbon atoms in the ring. Bicyclic heterocarbocyclic groups contain 8 to 12 carbon atoms, suitably 9 to 10 carbon atoms in the ring. Heterocarbocyclic groups may be substituted or unsubstituted. More than one substituent may be present. Substituents may also be themselves substituted. Suitable heterocarbocyclic groups include epoxy, tetrahydrofuranyl, azacyclopentyl, azacyclohexyl, piperidyl, and homopiperidyl. Heterocarbocyclic groups are not aromatic.

“Hydroxy” or “hydroxyl” means a chemical entity that consists of —OH. Alcohols contain hydroxy groups. Hydroxy groups may be free or protected. An alternative name for hydroxy is hydroxyl.

“Member atom” means a carbon, nitrogen, oxygen or sulfur atom. Member atoms may be substituted up to their normal valence. If substitution is not specified the substituents required for valency are hydrogen.

“Pharmaceutically acceptable carrier” means a carrier that is useful for the preparation of a pharmaceutical composition, i.e., generally compatible with the other ingredients of the composition, not deleterious to the recipient, and neither biologically nor otherwise undesirable. “A pharmaceutically acceptable carrier” includes both one and more than one carrier. Embodiments include carriers for topical, parenteral, intravenous, intraperitoneal intramuscular, sublingual, nasal and oral administration. “Pharmaceutically acceptable carrier” also includes agents for preparation of aqueous dispersions and sterile powders for injection or dispersions.

“Ring” means a collection of member atoms that are cyclic. Rings may be carbocyclic, aromatic, or heterocyclic or heteroaromatic, and may be substituted or unsubstituted, and may be saturated or unsaturated. More than one substituent may be present. Ring junctions with the main chain may be fused or spirocyclic. Rings may be monocyclic or bicyclic. Rings contain at least 3 member atoms and at most 10 member atoms. Monocyclic rings may contain 3 to 7 member atoms and bicyclic rings may contain from 8 to 12 member atoms. Bicyclic rings themselves may be fused or spirocyclic.

“Sulfonyl” refers to the —S(O)₂R′ group wherein R′ is alkoxy, alkyl, aryl, carbocyclic, heterocarbocyclic, heteroaryl, C₁₋₄ alkyl aryl or C₁₋₄ alkyl heteroaryl.

“Sulfonylamino” refers to the —S(O)₂NR′R′ group wherein each R′ is independently alkyl, aryl, heteroaryl, C₁₋₄ alkyl aryl or C₁₋₄ alkyl heteroaryl.

“Thioalkyl” refers to the group —S-alkyl.

“Thiol” refers to the group —SH.

Suitable substituents include, but are not limited to halogen, hydroxyl, alkoxy, alkyl, alkenyl, alkynyl, cycloalkyl, heterocycloalkyl, amino, amide, nitro, keto, oxo, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl and C₁₋₄ alkyl heteroaryl.

### Substituted Porphyrins

In one embodiment, the substituted porphyrins of the present invention have the structure shown in Formula I below:

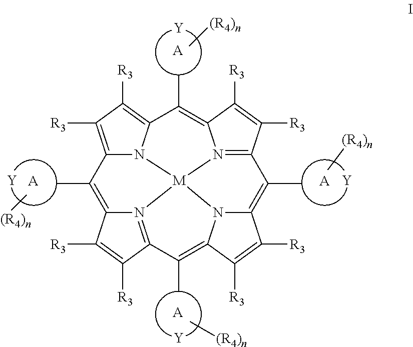

wherein each A is independently selected from the group consisting of an unsubstituted or substituted heteroaryl group and aryl group;
wherein each Y is independently selected from the group consisting of a CH and a heteroatom;
wherein each R₄ is independently —R₁—X—R₂;
wherein each R₁ is independently an unsubstituted or substituted alkylene;
wherein each X is independently selected from the group consisting of a direct bond and a heteroatom;
wherein each R₂ and R₃ are independently selected from the group consisting of hydrogen, halogen, hydroxyl, alkoxy, alkyl, alkenyl, alkynyl, cycloalkyl, heterocycloalkyl, amino, amide, nitro, keto, oxo, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl, and C₁₋₄ alkyl heteroaryl;
wherein each n is independently 0 to 2;
wherein M is selected from the group consisting of Mn, Fe, Co, Ni, Cu, V, and 2 hydrogens;
wherein at least one —R₁—X—R₂ contains at least one heteroatom; and
wherein at least one Y is N—R₄.

In some embodiments, the substituted porphyrins have enhanced lipophilicity. Lipophilicity can be measured via thin layer chromatography. The relative lipophilicity of a group of compounds can be determined in a given solvent system. Hydrophilic porphyrins will have an Rf of close to zero whereas the more lipophilic porphyrins have a larger Rf. The substituted porphyrins of the present invention suitably have a lipophilicity of at greater than zero in a solvent system of acetonitrile:water:KNO₃ (saturated aqueous) (8:1:1). Alternatively, the lipophilicity may be quantified by the standard octanol/water partition coefficient (log P). In some embodiments, the lipophilic substituted porphyrins have a greater bioavailability than their hydrophilic analogs. In addition, the lipophilic substituted porphyrins may have enhanced intracellular accumulation and/or intracellular uptake and/or potency in vivo as compared to hydrophilic analogs. Further, in other embodiments, the lipophilic substituted porphyrins may selectively target different cellular compartments, such as the mitochondria or the nucleus. Alternatively, other related cell/mitochondria viability assays (e.g. MTT) may be used.

In some embodiments, the substituted porphyrins of the present invention are less bulky and therefore are able to localize intracellularly. Alternatively, the nature of the substitutents, such as fluorine, hydroxy, or COO—, allows the substituted porphyrins to selectively localize intracellularly.

Suitably, the substituted porphyrins according to the present invention have one or more fluorines. Without wishing to be bound by theory, it is thought that the presence of fluoro groups enhance drug transport and potency.

In some embodiments, the substituted porphyrins have various active agents conjugated thereto. For example, anti-cancer agents, anti-inflammatory agents, analgesics (such as morphine), nitroxides, amino acids, peptides, peptidomimetics, antibodies, lipids or sugars may be conjugated to the substituted porphyrins.

Further, the substituted porphyrins of the present invention may be radiolabeled, for example with 18F. The radiolabeled compounds may then be used to determine biodistribution of the substituted porphyrins, such as with PET.

Compounds of the present invention may be obtained in the form of various salts or solvates. As the salts, ally acceptable salts or salts available as raw materials are used. In addition, all stereoisomers, enantiomers and diastereomers are contemplated.

Various substituted porphyrins contemplated by the present invention are shown in Table 1.

TABLE 1
Examples of substituted porphyrins according to the present invention

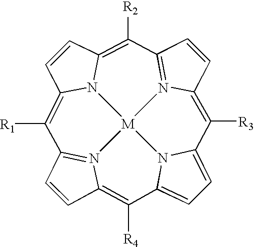

| | | | | trans- | | trans- | |
|---|---|---|---|---|---|---|---|
| | A4 | A₃B | cis-A₂B₂ | A₂B₂ | cis-A₂BC | A₂BC | ABCD |
| R₁ | Any group I | Any group 1 | Any group 1 | Any group 1 | Any group 1 | Any group 1 | Any group 1 |
| R₂ | Same as R₁ | Same as R₁ | Same as R₁ | Any group I different of R₁ | Same as R₁ | Any group I but R₂¹ R₁¹R₄ | Any group I but R²¹ R₁¹R₃¹ R₄ |
| R₃ | Same as R₁ | Same as R₁ | Any group I | Same as R₁ | Any group I but R₃¹ R₁¹R₄ | Same as R₁ | Any group I but R₃¹ R₁¹R₂¹ R₄ |
| R₄ | Same as R₁ | Any group II | Same as R₃ | Same as R₃ | Any group I but R₄¹ R₁¹ R₃ | Any group I but R₄¹ R₁¹R₂ | Any group I but R₄¹ R₁¹R₂¹ R₃ |
| Y₁—Y₅ | Any group II | Any group II | Any group II | Any group II | Any group II | Any group II | Any group II |
| Z | O, N, or S | O, N, or S | O, N, or S | O, N, or S | O, N, or S | O, N, or S | O, N, or S |
| R₅ | None or - R₇—X—R₈ | None or - R₇—X—R₈ | None or - R₇—X—R₈ | None or - R₇—X—R₈ | None or - R₇—X—R₈ | None or - R₇—X—R₈ | None or - R₇—X—R₈ |
| R₆ | None or - R₉—W—R₁₀ | None or - R₉—W—R₁₀ | None or - R₉—W—R₁₀ | None or - R₉—W—R₁₀ | None or - R₉—W—R₁₀ | None or - R₉—W—R₁₀ | None or - R₉—W—R₁₀ |
| R₇ | Any group III | Any group III | Any group III | Any group III | Any group III | Any group III | Any group III |
| X | None or Any group IV | None or Any group IV | None or Any group IV | None or Any group IV | None or Any group IV | None or Any group IV | None or Any group IV |
| R₈ | Any group II | Any group II | Any group II | Any group II | Any group II | Any group II | Any group II |
| R₉ | Any group III | Any group III | Any group III | Any group III | Any group III | Any group III | Any group III |
| W | None or Any group IV | None or Any group IV | None or Any group IV | None or Any group IV | None or Any group IV | None or Any group IV | None or Any group IV |
| R₁₀ | Any group II | Any group II | Any group II | Any group II | Any group II | Any group II | Any group II |

In all cases, M = Mn, Fe, Co, Ni Cu, V, or 2H.

Compounds included in Group I of Table 1 may include the following:

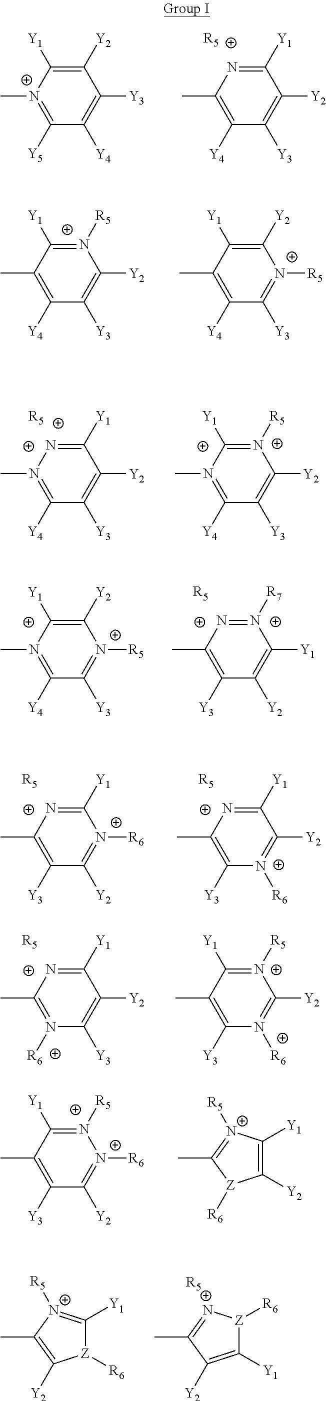

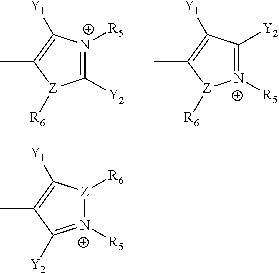

Substituents in Group II of Table 1 include —H, —F, —Cl, —Br, —I, —OH, —OR, —OCN, —NCO, —SH, —SR, —SNO, —SCN, —SSR, —S(O)R, —S(O)₂R, —SO₂R, —SO₂F, —SO₂Cl, —SO₂Cl, —SO₂Br, —SO₂I, —SO₂NH₂, —SO₂NHR, —SO₂NR₂, —SO₃, —SO₃R, —OSO₂R, —NCS, —NH₂, —NHR, —NR₂, —NC(O)H, —NC(O)R, —NC(S)H, —NC(S)R, —N═CH═, —N═CR₂, —N═NH, —N═NR, —NC, —NO, —NO₂, —CN, —CF₃, —C₁ to —C₁₈ saturated or unsaturated hydrocarbon chain, —C₃ to —C₈ saturated or unsaturated hydrocarbon cycle, —C(O)H, —C(O)R, —C(O)OH, —C(O)OR, —OC(O)R, —C(O)NH, —C(O)NR, —NC(O)H, —NC(O)R, —C(S)H, —C(S)R, —C(S)OH, —OC(S)H, —C(S)OR, —OC(S)R, —C(O)SH, —SC(O)H, —C(O)SR, —SC(O)R, —C(S)NH, —C(S)NH, —C(S)NR, -Ph, —C₆H₃R₂, —C₆H₂R₃, —C₆HR₄, —CR₅, —(CHₓF₂₋ₓ)ₙ[C(HₓF₃₋ₓ)]ₘ(n−1 to 20; x=0 to 2; m=0 to 20, —(CHₓCl₂₋ₓ)ₙ[C(HₓCl₃₋ₓ)]ₘ (n=1 to 20; x=0 to 2; m=0 to 20), —(CHₓBr₂₋ₓ)ₘ (n=1 to 20; x=0 to 2; m=0 to 20), —PR₂, —PR₃, —P(O)R₂, and —P(S)R₂. Compounds included in Group II include those wherein R=any group II (preferably, —H, —F, —Cl, —Br, −1, —OH, —OCN, —NCO, —SH, —NCO, —NH₂, —NO₂, —CN, —CF₃, —C₁ to —C₁₈ saturated or unsaturated hydrocarbon chain, —C₃ to —C₈ saturated or unsaturated hydrocarbon cycle, —C(O)H, —C(O)OH, —C(O)NH, —NC(O)H, —C(S)NH, -Ph, —C₆H₄R, —C₆H₂R₃, —C₆HR₄, —CR₅, —(CHₓF₂₋ₓ)ₘ[C(HₓF₃₋ₓ)]ₘ (n=1 to 20; x=0 to 2; m=0 to 20), —(CHₓCl₂₋ₓ)ₙ[C(HₓCl₃₋ₓ)]ₘ, (n=1 to 20; x=0 to 2; m=0 to 20), —(CHₓBr₂₋ₓ)ₙ[C(HₓBr₃₋ₓ)]ₘ (n=1 to 20; x=0 to 2; m=0 to 20)], amino acid, peptide, protein, carbohydrate, fatty acid, nucleic acid, ribonucleic acid, nucleic bases, nucleoside, nucleotide, vitamin, opioid, virus, surfactant, nitroxide, lipid, enzyme, denaturated enzyme, co-enzyme, cancer drug, cancer pro-drug, hormone, glycoprotein, lipoprotein, glycolipid, phopholipid, cholesterol, quinone, ubiquinone, Shiff base, anti-inflammatory drug, analgesic drug, anesthetic drug, flavonoides, or flavones.

Substituents in Group III of Table 1 include —(CH₂)ₙ— (n=1 to 100), —(CHOH)ₙ— (n=1 to 100), —(CHOR)ₙ— (n=1 to 100), —[(CH₂)ₙO]ₘCH₂—(n=1 to 100; m=1 to 100), —[(CH₂)ₙS]ₘCH₂— (n=1 to 100; m=1 to 100), —[(CH₂)ₘS(O)₂]ₘCH₂— (n=1 to 100; m=1 to 100), —[(CH₂)nS(O)₂]ₘCH₂— (n=1 to 100), —[(CH₂)ₙNH]ₘCH₂— (n=1 to 100), —[(CH₂)ₙNR]ₘCH₂— (n=1 to 100; m=1 to 100), —[(CH₂)ₙ(C₆H₄)—Iₘ-(n=1 to 100; m=1 to 100), and —[(CH₂)ₙ(C₆H₄)—IₘCH₂— (n=1 to 100; m=1 to 100). Compounds included in Group III include those wherein R=any group II (preferably, —H, —F, —Cl, —Br, —I, —OH, OCN, —NCO, —SH, —NCS, —NH₂, —NO₂, —CN, —CF₃, —C₁ to —C₁₈ saturated or unsaturated hydrocarbon chain, —C₃ to —C₈ saturated or unsaturated hydrocarbon cycle, —C(O)H, —C(O)OH, —C(O)NH, —NC(O)H, —C(S)NH, -Ph, —C₆H₄R, —C₆H₃R₂, —C₆H₂R₃, —C₆HR₄, —CR₅, —(CHₓF₂₋ₓ)ₐ[C(HₓF₃₋ₓ)]ₘ (n=1 to 20; x=0 to 2; m=0 to 20), —(CHₓCl₂₋ₓ)ₙ[C(HₓCl₃₋ₓ)]ₘ (n=1 to 20; x=0 to 2; m=0 to 20), —(CHₓBr₂₋ₓ)ₙ[C(HₓBr₃₋ₓ)]ₘ (n=1 to 20; x=0 to 2; m=0 to 20)], amino acid, peptide, protein, carbohydrate, fatty acid, nucleic acid, ribonucleic acid, nucleic bases, nucleoside, nucleotide, vitamin opioid, virus, surfactant, nitroxide, lipid, enzyme, denaturated enzyme, co-enzyme, cancer drug, cancer pro-drug, hormone, glycoprotein, lipoprotein, glycolipid, phopholipid, cholesterol, quinone, ubiquinone, Shiff base, anti-inflammatory drug, analgesic drug, anesthetic drug, flavonoides, or flavones.

Substituents in Group IV of Table 1 include —O—, —NH—, —NR—, —S—, —SS—, —SSS—, —S(O)—, —S(O)₂—, —C(O)—, —OS(O)₂—, —S(O)₂O—, —PR—, —PR₂—, —P(O)R—, and —P(S)R—. Compounds included in Group IV include those wherein R=any group II (preferably, —H, —F, —Cl, —Br, —I, —OH, —OCN, —NCO, —SH, —NCS, —NH₂, —NO₂, —CN, —CF₃, —C₁, to —C₁₋₈ saturated or unsaturated hydrocarbon chain, —C₃ to —C₈ saturated or unsaturated hydrocarbon cycle, —C(O)H, —C(O)OH, —C(O)NH, —NC(O)H, —C(S)NH, -Ph, —C₆H₄R, —C₆H₃R₂, —C₆H₂R₃, —C₆HR₄, —CR₅, —(CHₓ(F₂₋ₓ)ₙ[C(HₓF₃₋ₓ)]ₘ (n=1 to 20; x=0 to 2; m=0 to 20), —(CHₓCl₂₋ₓ)ₙ[C(HₓCl₃₋ₓ)]ₘ (n=1 to 20; x=0 to 2; m=0 to 20), —(CHₓBr₂₋ₓ)ₙ[C(HₓBr₃₋ₓ)ₘ (n=1 to 20; x=0 to 2; m=0 to 20)], amino acid, peptide, protein.

### Synthesis of Substituted Porphyrins

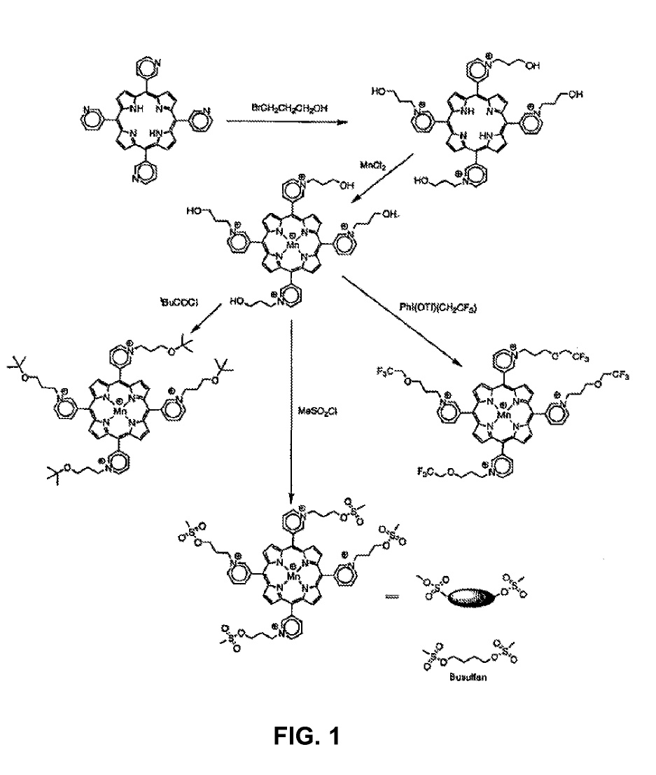

The substituted porphyrins of the present invention may be synthesized according to the schemes shown in FIG. 1.

For example, the ortho isomeric substituted Mn pyridylporphyrins are synthesized through several steps. In a first step, an aldehyde and a pyrrole are condensed in a heated carboxylic acid, such as propionic acid at 130° C., to give a metal-free non-substituted porphyrinogen which in the presence of oxidant (H₂O₂ or O₂) gets oxidized to porphyrin.

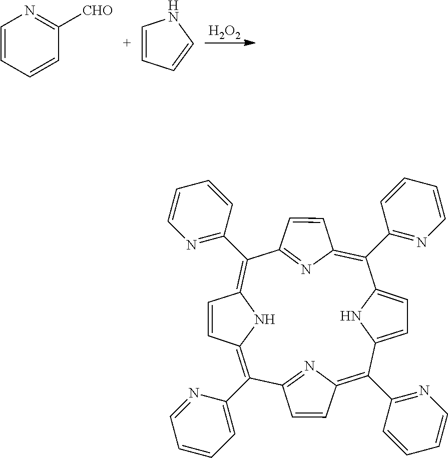

The product, H₂T-2-PyP is chromatographed using dichloromethane/methanol solvent system and is then forwarded to a second step where the pyridyl nitrogens are derivatized with appropriate side chains, such as ethyl. The derivatization/quaternization occurs at ˜100° C. for a certain time period with p-alkyl(or derivatized alkyl)toluenesulfonate, e.g. p-ethyltoluenesulfonate (time period depending upon the length and bulkiness of the alkyl or derivatized alkyl). The completion is followed by TLC in a solvent system 80:10:10 (acetonitrile:KNO₃(aq. saturated):H₂O), until single spot is obtained (with longer chains the atropoisomers will emerge and multiple spots will be seen). Whether atropoisomers are resolved or incomplete quaternization occurs may be witnessed by mass spectrometry. The mixture is then washed with chloroform and water in a separatory funnel to remove toluenesulfonate and DMF. The aqueous phase is used to isolate the chloride salt as described below.

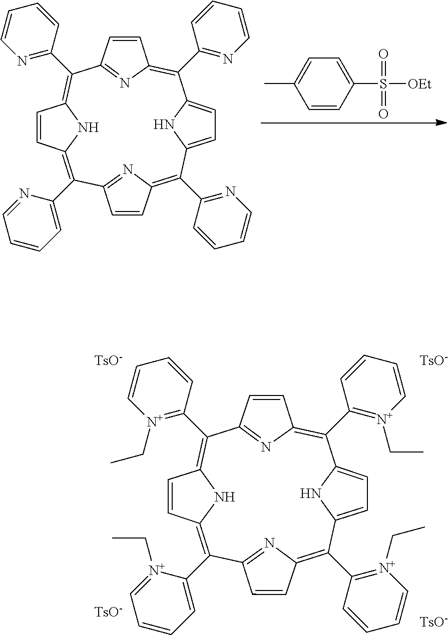

In aqueous phase porphyrin is precipitated first from water with NH₄ PF₆ as PF₆⁻ salt, and washed extensively with diethylether. The PF₆⁻ salt was then dissolved in acetone and then the chloride salt is precipitated from acetone with tetrabutylammonium chloride and washed thoroughly with acetone.

In a third step the insertion of Mn is done in aqueous solution upon increasing pH to 12.3 with 20-fold excess MnCl₂. The completion can be checked by uv/vis and by TLC (same solvent as above) (as the absence of the fluorescent spot of meal-free porphyrin). The excess of Mn (as hydroxo/oxo complexes) is removed by double filtration (over filter paper) and then the Mn porphyrin is precipitated first as PF₆⁻ salt from water, (depicted below) and then as chloride salt from acetone as described above for the metal-free ligand. The precipitation is done twice to assure the full removal of the water-soluble low-molecular weight Mn complexes.

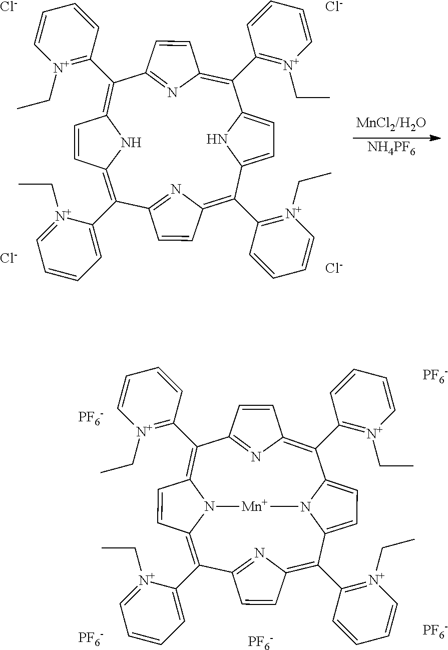

### Methods of Using Substituted Porphyrins

The substituted porphyrins may be used to treat various conditions, including those resulting, at least in part, from oxidative stress injury (damage resulting from excessive levels of reactive oxygen and nitrogen species). In other embodiments, the substituted porphyrins of the present invention reduce oxidative stress. Porphyrins are effective functional catalytic antioxidants, modulators of redox-signaling pathways, potent radioprotectors, and anti-cancer agents. Again, without wishing to be bound by theory, it is thought that the antioxidant properties of porphyrins stem from their ability to regulate redox-active transcription factors via modulation of reactive oxygen and nitrogen species (ROS/RNS) and/or their ability to decrease biological damage by directly scavenging those species.

Pathological conditions that may be treated by the substituted porphyrins according to the present invention include, but are not limited to, central nervous system, injuries (such as ALS, Alzheimer's, multiple sclerosis, Parkinson's, etc.), stroke, spinal cord injury, cancer, ischemia/reperfusion injuries, cardiovascular injuries, arthritis, sickle cell disease, radiation injury, auto-immune diseases, diabetes, morphine tolerance, drug dependence/addiction and inflammatory conditions.

In a further embodiment, substituted porphyrins according to the present invention are suitable for use as anti-cancer agents in cancers such as, lung, breast, brain, skin, head and neck, prostate, pancreas, gastrointestinal, and colon. Without wishing to be bound by theory, it is thought that the anticancer activity of the substituted porphyrins arises from the redox-based impact of the substituted porphyrins on oxidative stress and thus on the HIF/VEGF/NOS pathways. In some embodiments, the substituted porphyrins suppress angiogenesis in tumors.

In other embodiments, the substituted porphyrins are potent adjuvants in radiation therapy, hyperthermia, chemotherapy and pain management, such as morphine tolerance reversal. The substituted porphyrins can be administered in combination with other active agents, such as anti-cancer agents (e.g. gleevac, cisplatin, taxol, vincristine, doxorubicin, cyclophosphamide, statins, melphalan, fludarabine, camptotechins, monoclonal and polyclonal antibodies against VEG, VEGFr, EGF, ERGFr, etc.), anti-inflammatory agents (e.g. cyclooxygenase inhibitors, NOS inhibitors, NADPH oxidase inhibitors, etc.), and analgesics (e.g. morphine, codeine, aspirin, acetaminophen, ibuprofen, etc.).

Thus, a synergistic effect may be seen where a substituted porphyrin of the present invention is administered in combination with an anti-cancer agent, whether or not the substituted porphyrin is conjugated to an anti-cancer agent. If a substituted porphyrin conjugated to an anti-cancer agent is administered in combination with a second anti-cancer agent, the second anti-cancer agent may be the same or different as the conjugated anti-cancer agent.

In one embodiment, a cell is contacted with an amount of a substituted porphyrin effective to reduce oxidative stress. Suitably, the reduction in oxidative stress can be measured by measuring a reduction in the amount of reactive oxygen and/or nitrogen species. The term “contacting a cell” is used to mean contacting a cell in vitro or in vivo (i.e. in a subject, such as a mammal, including humans, rabbits, cats and dogs). In one embodiment, the cell may contacted as a result of administration of a substituted porphyrin to a subject.

An effective amount of a substituted porphyrin according to the present invention will vary with the particular condition being treated, the age and physical condition of the subject being treated, the severity of the condition, the duration of treatment, the nature of concurrent therapy, the route of administration, the particular pharmaceutically-acceptable carrier utilized, and like factors within the knowledge and expertise of the attending physician. For example, an effective amount of the substituted porphyrins of the present invention for systemic administration is from about 0.01 to about 100 mg/kg body weight, preferably from about 0.1 to about 100 mg/kg per body weight, most preferably from about 1 to about 50 mg/kg body weight per day. Transdermal dosages would be designed to attain similar serum or plasma levels, based upon techniques known to those skilled in the art of pharmacokinetics and transdermal formulations. Plasma levels for systemic administration are expected to be in the range of 0.001 to 100 microgram/mL, more preferably from 0.01 to 50 microgram/mL and most preferably from 0.1 to 10 microgram/mL. While these dosages are based upon a daily administration rate, the substituted porphyrins of the present invention may also be administered at other intervals, such as twice per day, twice weekly, once weekly, or once a month. The substituted porphyrins of the present invention may also be administered in a continuous mode, for example, using an osmotic pump. In one embodiment, the porphyrins may be initially administered more frequently (e.g. daily) at higher doses to establish a loading dose with continued administration at a lower less frequent dose. One of ordinary skill in the art would be able to calculate suitable effective amounts for other intervals of administration. For example, the efficacy of various substituted porphyrins in vivo is affected by both the antioxidant potency of the substituted porphyrin and the bioavailability of that porphyrin.

The additional active agent or agents can be administered simultaneously or sequentially with the substituted porphyrins of the present invention. Sequential administration includes administration before or after the substituted porphyrins of the present invention. In some embodiments, the additional active agent or agents can be administered in the same composition as the substituted porphyrins of the present invention. In other embodiments, there can be an interval of time between administration of the additional active agent and the substituted porphyrins of the present invention.

In some embodiments, the administration of an additional therapeutic agent with a compound of the present invention will enable lower doses of the other therapeutic agents to be administered for a longer period of time.

### Compositions Comprising Substituted Porphyrins

In one embodiment, the substituted porphyrins are administered in a pharmaceutically acceptable composition, such as in or with a pharmaceutically acceptable carrier.

Compositions may include one or more of the isoforms of the substituted porphyrins of the present invention. When racemates exists, each enantiomer or diastereomer may be separately used, or they may be combined in any proportion. Where tautomers exist all possible tautomers are specifically contemplated.

Pharmaceutical compositions for use in accordance with the present invention may be formulated in a conventional manner using one or more physiologically acceptable carriers or excipients. Thus, the substituted porphyrins may be formulated for administration by, for example, solid dosing, eyedrop, in a topical oil-based formulation, injection, inhalation (either through the mouth or the nose), implants, or oral, buccal, parenteral or rectal administration. Techniques and formulations may generally be found in “Remington's Pharmaceutical Sciences”, (Meade Publishing Co., Easton, Pa.).

The route by which the substituted porphyrins of the present invention (component A) will be administered and the form of the composition will dictate the type of carrier (component B) to be used. The composition may be in a variety of forms, suitable, for example, for systemic administration (e.g., oral, rectal, nasal, sublingual, buccal, implants, or parenteral) or topical administration (e.g., local application on the skin, liposome delivery systems, or iontophoresis).

Carriers for systemic administration typically comprise at least one of a) diluents, b) lubricants, c) binders, d) disintegrants, e) colorants, f) flavors, g) sweeteners, h) antioxidants, j) preservatives, k) glidants, m) solvents, n) suspending agents, o) wetting agents, p) surfactants, combinations thereof, and others. All carriers are optional in the systemic compositions.

Ingredient a) is a diluent. Suitable diluents for solid dosage forms include sugars such as glucose, lactose, dextrose, and sucrose; diols such as propylene glycol; calcium carbonate; sodium carbonate; sugar alcohols, such as glycerin, mannitol, and sorbitol. The amount of ingredient a) in the systemic or topical composition is typically about 50 to about 90%.

Ingredient b) is a lubricant. Suitable lubricants for solid dosage forms are exemplified by solid lubricants including silica, talc, stearic acid and its magnesium salts and calcium salts, calcium sulfate; and liquid lubricants such as polyethylene glycol; and vegetable oils such as peanut oil, cottonseed oil, sesame oil, olive oil, corn oil and oil of theobroma. The amount of ingredient b) in the systemic or topical composition is typically about 5 to about 10%.

Ingredient c) is a binder. Suitable binders for solid dosage forms include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of ingredient c) in the systemic composition is typically about 5 to about 50%.

Ingredient d) is a disintegrant. Suitable disintegrants for solid dosage forms include agar, alginic acid and the sodium salt thereof, effervescent mixtures, croscarmellose, crospovidone, sodium carboxymethyl starch, sodium starch glycolate, clays, and ion exchange resins. The amount of ingredient d) in the systemic or topical composition is typically about 0.1 to about 10%.

Ingredient e) for solid dosage forms is a colorant such as an FD&C dye. When used, the amount of ingredient e) in the systemic or topical composition is typically about 0.005 to about 0.1%.

Ingredient f) for solid dosage forms is a flavor such as menthol, peppermint, and fruit flavors. The amount of ingredient f), when used, in the systemic or topical composition is typically about 0.1 to about 1.0%.

Ingredient g) for solid dosage forms is a sweetener such as aspartame and saccharin. The amount of ingredient g) in the systemic or topical composition is typically about 0.001 to about 1%.

Ingredient h) is an antioxidant such as butylated hydroxyanisole (“BHA”), butylated hydroxytoluene (“BHT”), and vitamin E. The amount of ingredient h) in the systemic or topical composition is typically about 0.1 to about 5%.

Ingredient j) is a preservative such as benzalkonium chloride, methyl paraben and sodium benzoate. The amount of ingredient j) in the systemic or topical composition is typically about 0.01 to about 5%.

Ingredient k) for solid dosage forms is a glidant such as silicon dioxide. The amount of ingredient k) in the systemic or topical composition is typically about 1 to about 5%.

Ingredient m) is a solvent, such as water, isotonic saline, ethyl oleate, glycerine, hydroxylated castor oils, alcohols such as ethanol, and phosphate buffer solutions. The amount of ingredient m) in the systemic or topical composition is typically from about 0 to about 100%.

Ingredient n) is a suspending agent. Suitable suspending agents include Avicel® RC-591 (from FMC Corporation of Philadelphia, Pa.) and sodium alginate. The amount of ingredient n) in the systemic or topical composition is typically about 1 to about 8%.

Ingredient o) is a surfactant such as lecithin, Polysorbate 80, and sodium lauryl sulfate, and the TWEENS® from Atlas Powder Company of Wilmington, Del. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of ingredient o) in the systemic or topical composition is typically about 0.1% to about 5%.

Although the amounts of components A and B in the systemic compositions will vary depending on the type of systemic composition prepared, the specific derivative selected for component A and the ingredients of component B, in general, system compositions comprise about 0.01% to about 50% of component A and about 50% to about 99.99% of component B.

Compositions for parenteral administration typically comprise A) about 0.01 to about 10% of the substituted porphyrins of the present invention and B) about 90 to about 99.99% of a carrier comprising a) a diluent and m) a solvent. In one embodiment, component a) comprises propylene glycol and m) comprises ethanol or ethyl oleate.

Compositions for oral administration can have various dosage forms. For example, solid forms include tablets, capsules, granules, and bulk powders. These oral dosage forms comprise a safe and effective amount, usually at least about 5%, and more particularly from about 25% to about 50% of component A). The oral dosage compositions further comprise about 50 to about 95% of component B), and more particularly, from about 50 to about 75%.

Tablets can be compressed, tablet triturates, enteric-coated, sugar-coated, film-coated, or multiple-compressed. Tablets typically comprise component A, and component B a carrier comprising ingredients selected from the group consisting of a) diluents, b) lubricants, c) binders, d) disintegrants, e) colorants, f) flavors, g) sweeteners, k) glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain g) sweeteners such as aspartame and saccharin, or f) flavors such as menthol, peppermint, fruit flavors, or a combination thereof.

Capsules (including implants, time release and sustained release formulations) typically comprise component. A, and a carrier comprising one or more a) diluents disclosed above in a capsule comprising gelatin. Granules typically comprise component A, and preferably further comprise k) glidants such as silicon dioxide to improve flow characteristics. Implants can be of the biodegradable or the non-biodegradable type. Implants may be prepared using any known biocompatible formulation.

The selection of ingredients in the carrier for oral compositions depends on secondary considerations like taste, cost, and shelf stability, which are not critical for the purposes of this invention. One skilled in the art would know how to select appropriate ingredients without undue experimentation.

The solid compositions may also be coated by conventional methods, typically with pH or time-dependent coatings, such that component A is released in the gastrointestinal tract in the vicinity of the desired application, or at various points and times to extend the desired action. The coatings typically comprise one or more components selected from the group consisting of cellulose acetate phthalate, polyvinyl acetate phthalate, hydroxypropyl methyl cellulose phthalate, ethyl cellulose, EUDRAGIT® coatings (available from Rohm & Haas G.M.B.H. of Darmstadt, Germany), waxes and shellac.

Compositions for oral administration can also have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically comprise component A and component B, namely, a carrier comprising ingredients selected from the group consisting of a) diluents, e) colorants, f) flavors, g) sweeteners, j) preservatives, m) solvents, n) suspending agents, and o) surfactants. Peroral liquid compositions preferably comprise one or more ingredients selected from the group consisting of e) colorants, f) flavors, and g) sweeteners.

Other compositions useful for attaining systemic delivery of the subject substituted porphyrins include sublingual, buccal and nasal dosage forms. Such compositions typically comprise one or more of soluble filler substances such as a) diluents including sucrose, sorbitol and mannitol; and c) binders such as acacia, microcrystalline cellulose, carboxymethyl cellulose, and hydroxypropyl methylcellulose. Such compositions may further comprise b) lubricants, e) colorants, f) flavors, g) sweeteners, h) antioxidants, and k) glidants.

In one embodiment of the invention, the substituted porphyrins of the present invention are topically administered.

Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions comprise: component A, the substituted porphyrins described above, and component B, a carrier. Component B may further comprise one or more optional components.

The exact amounts of each component in the topical composition depend on various factors. The amount of component A added to the topical composition is dependent on the IC50 of component A, typically expressed in nanomolar (nM) units. For example, if the IC50 of the medicament is 45 nM, the amount of component A will be from about 0.04 to about 4%. If the IC50 of the medicament is 100 nM, the amount of component A) will be from about 0.08 to about 8%. If the IC50 of the medicament is 1000 nM, the amount of component A will be from about 0.8 to about 80%. If the amount of component A is outside the ranges specified above (i.e., lower), efficacy of the treatment may be reduced. One skilled in the art understands how to calculate and understand an IC50. The remainder of the composition, up to 100%, is component B.

The amount of the carrier employed in conjunction with component A is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

Component B may comprise a single ingredient or a combination of two or more ingredients. In the topical compositions, component B comprises a topical carrier. Suitable topical carriers comprise one or more ingredients selected from the group consisting of phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols and symmetrical alcohols.

The carrier of the topical composition may further comprise one or more ingredients selected from the group consisting of q) emollients, r) propellants, s) solvents, t) humectants, u) thickeners, v) powders, w) fragrances, x) pigments, and y) preservatives.

Ingredient q) is an emollient. The amount of ingredient q) in a skin-based topical composition is typically about 5 to about 95%. Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane.

Ingredient r) is a propellant. The amount of ingredient r) in the topical composition is typically about 0 to about 95%. Suitable propellants include propane, butane, isobutane, dimethyl ether, carbon dioxide, nitrous oxide, and combinations thereof.

Ingredient s) is a solvent. The amount of ingredient s) in the topical composition is typically about 0 to about 95%. Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols.

Ingredient t) is a humectant. The amount of ingredient t) in the topical composition is typically 0 to 95%. Suitable humectants include glycerin, sorbitol, sodium 2-pyrrolidone-5-carboxylate, soluble collagen, dibutyl phthalate, gelatin, and combinations thereof. Specific humectants include glycerin.

Ingredient u) is a thickener. The amount of ingredient u) in the topical composition is typically about 0 to about 95%.

Ingredient v) is a powder. The amount of ingredient v) in the topical composition is typically 0 to 95%. Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof.

Ingredient w) is a fragrance. The amount of ingredient w) in the topical composition is typically about 0 to about 0.5%, particularly, about 0.001 to about 0.1%.

Ingredient x) is a pigment. Suitable pigments for skin applications include inorganic pigments, organic lake pigments, pearlescent pigments, and mixtures thereof. Inorganic pigments useful in this invention include those selected from the group consisting of rutile or anatase titanium dioxide, coded in the Color Index under the reference CI 77,891; black, yellow, red and brown iron oxides, coded under references CI 77,499, 77,492 and, 77,491; manganese violet (CI 77,742); ultramarine blue (CI 77,007); chromium oxide (CI 77,288); chromium hydrate (CI 77,289); and ferric blue (CI 77,510) and mixtures thereof.

The organic pigments and lakes useful in this invention include those selected from the group consisting of D&C Red No. 19 (CI 45,170), D&C Red No. 9 (CI 15,585), D&C Red No. 21 (CI 45,380), D&C Orange No. 4 (CI 15,510), D&C Orange No. 5 (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430), the dye or lakes based on Cochineal Carmine (CI 75,570) and mixtures thereof.

The pearlescent pigments useful in this invention include those selected from the group consisting of the white pearlescent pigments such as mica coated with titanium oxide, bismuth oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof. The amount of pigment in the topical composition is typically about 0 to about 10%.

Component A may be included in kits comprising component A, a systemic or topical composition described above, or both; and information, instructions, or both that use of the kit will provide treatment for cosmetic and medical conditions in mammals (particularly humans). The information and instructions may be in the form of words, pictures, or both, and the like. In addition or in the alternative, the kit may comprise the medicament, a composition, or both; and information, instructions, or both, regarding methods of application of medicament, or of composition, preferably with the benefit of treating or preventing cosmetic and medical conditions in mammals (e.g., humans).

### EXAMPLES

The present invention is further described in the following non-limiting examples.

### Example 1

### Synthesis of MnTnPrOH-3-PyP⁵⁺

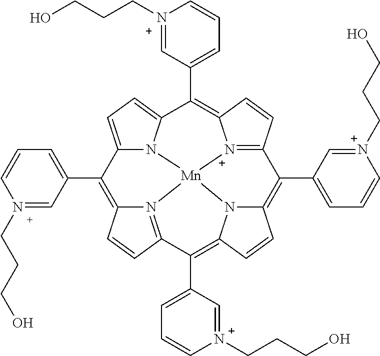

0.05 g of H2T-3-PyP (Frontier Scientific) and 2 ml of 3-bromo-1-propanol (Sigma) was dissolved in 25 ml DMF and heated at 100° C. in round bottom flask equipped with reflux condenser for 48 hours. In last hour temperature was increased up to 160° C. Heating was discontinued and solution was left at room temperature overnight. Brownish precipitate was centrifuged and washed first 5 times with ether and then 5 times with acetone. DMF supernatant was precipitated in portions of 1 ml with 12-14 ml ether, centrifuged and washed with ether 5 times. Both precipitates were left in vacuum at room temperature overnight. Later TLC analysis revealed that precipitate from DMF was dirty and contained low level of porphyrin. Brownish precipitate was dissolved in about 40 ml of H2O and precipitated with NH4 PF6 and washed with ether. Precipitate was dissolved in acetone and filtered. Porphyrin was precipitated with tetrabutylammonium chloride (TBACl), filtered and washed with acetone. Precipitation from water and acetone was repeated. Filtered product was left in vacuum overnight at room temperature. The uv/vis spectrum is as expected based on published data on analogous compounds (Batinic-Haberle et al., Inorg. Chem. 1999, 38, 4011-4022).

Porphyrin is metallated with 20-fold excess MnCl₂ upon the increase of pH to ˜12 to deprotonate pyrrolic nitrogens. The solution was then filtered and Mn porphyrin precipitated as described above.

Thin layer chromatography (TLC) was carried out in polyester-backed silica-gel sheets without fluorescent indicator (Sigma-Aldrich Z122777-25EA) cut into ˜2×20 cm strips and eluted with a saturated KNO₃(aq):H₂O:MeCN (1:1:8, v/v/v) mixture in a capped 1 L chamber. Typically, 1 microliter of ˜1 mM samples were applied at ˜1 cm of the strip border and the solvent front was allowed to run ˜12 cm.

UV-vis spectra were recorded in H₂O on a Shimadzu UV-2501PC spectrophotometer (0.5 nm resolution).

### Example 2

### Synthesis of MnTnPrOH-2-PyP⁵⁺

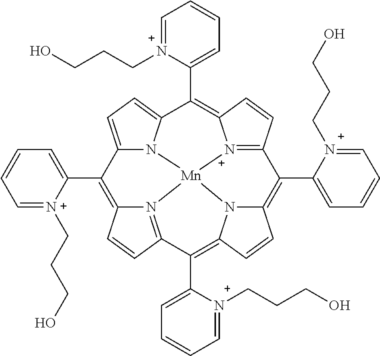

0.0515 g of H2T-2-PyP (Frontier Scientific) and 3 ml of 3-bromo-1-propanol (Sigma) was dissolved in 10 ml DMF and heated at 105° C. in round bottom flask equipped with reflux condenser for 48 hours. Reaction was followed up with TLC. Solution was transferred in Kimax centrifuge tube in portions of 1 ml and precipitated with addition of ether. Precipitate was washed with ether 5 times, suspending precipitate in ether by vortexing and sonification followed by centrifugation. Then precipitate was washed 5 times with 10 ml portions of CH₂Cl₂ and again 5 times with 10 ml portions of ether and then dissolved in water (15 ml). Porphyrin was precipitated with NH₄ PF₆. The gummy precipitate was dissolved in acetone and precipitated with addition of TBACl, dissolved in H₂O and precipitated with NH₄ PF₆, filtered and washed with ether (precipitate was very gummy and sticky). After washing with ether precipitate was dissolved in acetone and precipitated with TBACl and washed with acetone. Then precipitate was dissolved in 20 ml H₂O and precipitated with NH₄ PF₆, filtered and washed with ether and again dissolved in acetone and precipitated with TBACl and washed with acetone. Precipitate was left in vacuum at room temperature over night.

The compound is metallated and characterized as described for meta analogue and used for SOD-deficient E. coli aerobic growth in same experiments as described above for meta analogue. The data show that several meta and ortho isomers bearing the same alkyl chain, although having markedly different antioxidant properties in aqueous solution, provide identical protection in the E. coli model. (See Example 9 below).

### Example 3

### Mn(Et-X-Py)nPh₄₋ₙPₙ₊₁ (X=2, or 3; n=1, 2, or 3)

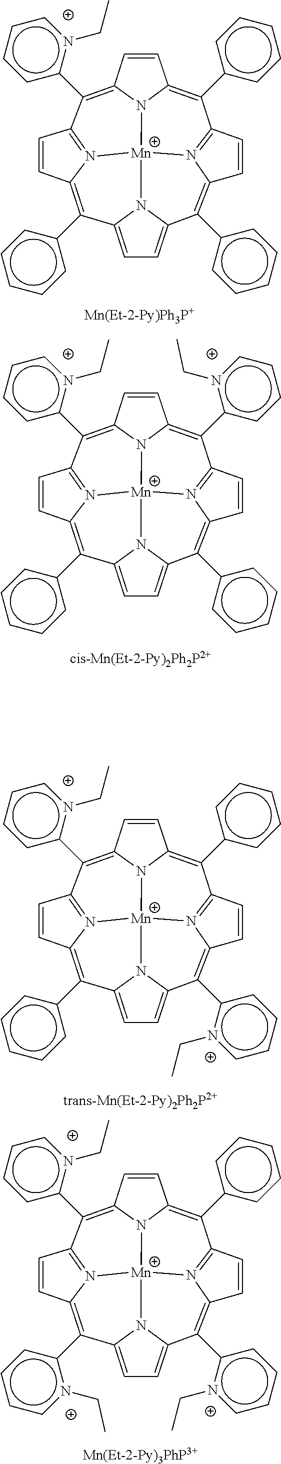

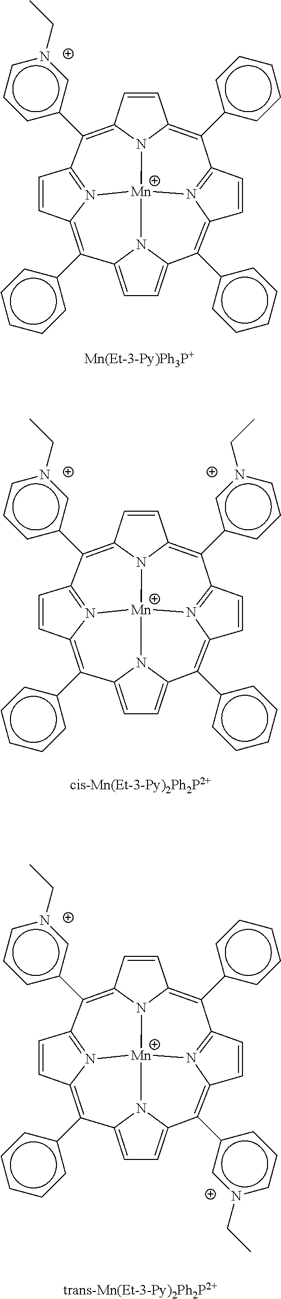

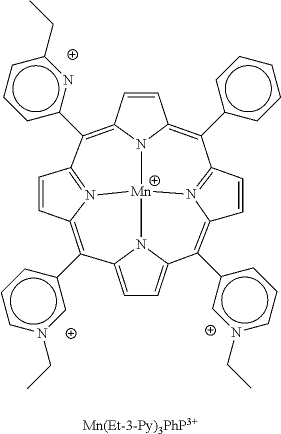

The free-base H₂(X-Py)ₙPh₄₋ₙPₙ₊₁ (X=2, or 3; n=1, 2, or 3) is prepared by the stoichiometric condensation of pyrrole, benzaldehyde, and the corresponding ortho-(X=2) or meta-(X=3) isomer of carboxypyridine aldehyde as described by Sari et al. (Sari et al, Biochemistry 1990, 29, 4205-4215). The alkylation of the pyridyl moieties of H₂(X-Py)ₙPh₄₋ₙPₙ₊₁ (X=2, or 3; n=1, 2, or 3) with ethyl 4-toluenesulfonate to yield H₂(Et-X-Py)ₙPh₄₋ₙPₙ₊₁ (X=2, or 3; n=1, 2, or 3) is carried out in DMF at 105° C. using a procedure adapted from that described to the preparation of the MnTE-2-PyP analogue (Batinic-Haberle et al, Inorg. Chem. 1999, 38, 4011-4022). The cationic derivatives are metallated, characterized and used for SOD-deficient E. coli aerobic growth in experiments analogous to that described for the compound in Example 1.

### Example 4

### Synthesis of MnTtBuCO₂(CH₂)₃-3-PyP⁵⁺

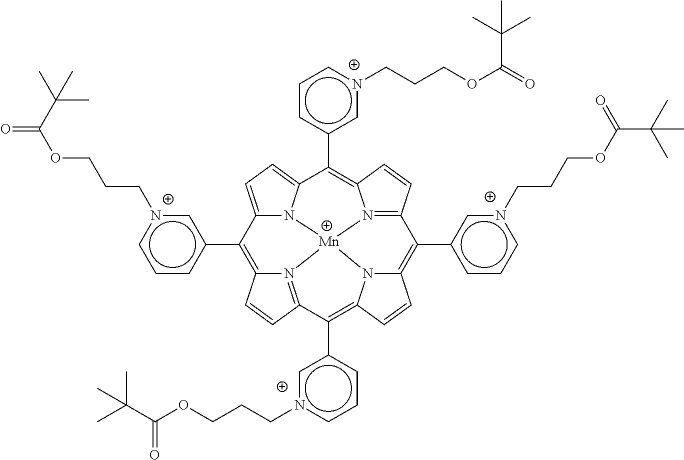

The acylation of MnTnPrOH-3-PyP⁵⁺ is carried out following standard acoholysis of acyl halide procedures (Smith, M. B and March, J., in: March's Advanced Organic Chemistry, 6^{th} Edition, John Wiley and Sons: Hoboken, 2007, pp. 1411-1412). MnTnPrOH-3-PyP⁵⁺, anhydrous pyridine (catalyst, 10 equiv.), and pivaloyl chloride (50 equiv.) are dissolved in anhydrous DMF and heated for 48 hours to 105° C. in a round bottom flask equipped with a reflux condenser and protected from moisture. The progress of the reaction is monitored by TLC. The product is purified using a work-up procedure similar to that described in Example 1.

### Example 5

### Synthesis of MnTMeSO₃(CH₂)₃-3-PyP⁵⁺

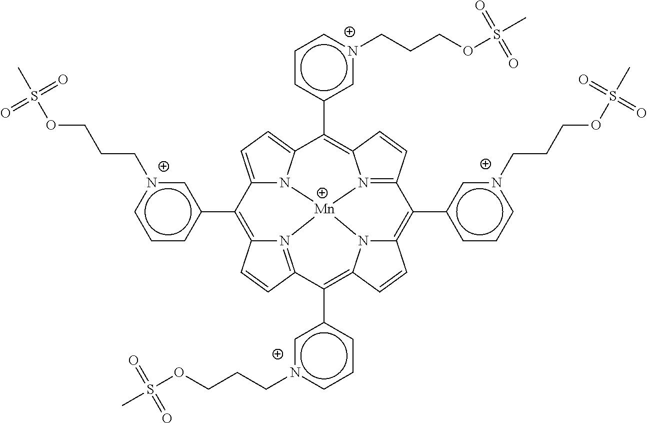

The sulfonylation of MnTnPrOH-3-PyP⁵⁺ follows a standard procedure for acoholysis of sulfonyl halides (Smith, M. B and March, J., in: March's Advanced Organic Chemistry, 6^{th} Edition, John Wiley and Sons: Hoboken, 2007, p. 1473). MnTnPrOH-3-PyP⁵⁺, anhydrous pyridine (catalyst, 10 equiv.), and methanesulfonyl chloride (50 equiv.) are dissolved in anhydrous DMF and heated for 48 hours to 105° C. in a round bottom flask equipped with a reflux condenser and protected from moisture. The progress of the reaction is monitored by TLC. The product is purified using a work-up procedure similar to that described in Example 1.

### Example 6

### Synthesis of MnTCF₃CH₂O(CH₂)₃-3-PyP⁵⁺

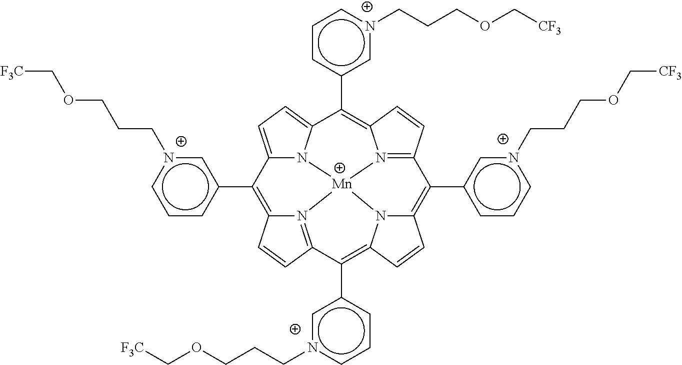

The fluoroalkylation of MnTnPrOH-3-PyP⁵⁺ is carried out using an appropriate fluoroalkylating agent, such as (1,1-dihydroperfluoroalkyl)phenyliodonium triflates (Umemoto, T. and Gotoh, Y., Bull. Chem. Soc. Jpn. 1987, 60, 3307-3313) and following a procedure adapted from that described by Umemoto and Gotoh (Umemoto, T. and Gotoh, Y., J. Fluorine Chem. 1986, 31, 231-236). The alkoxyde derivative of MnTnPrOH-3-PyP⁵⁺ is prepared via alcohol deprotonation with lithium hydride (10 equiv.) in anhydrous DMF. To this mixture, is added (2,2,2-trifluoroethane)phenyliodonium triflate (50 equiv.) and the system is heated for 24 hours at 50° C., protected from moisture. The progress of the reaction is monitored by TLC. The product is purified using a work-up procedure similar to that described in Example 1.

### Example 7

### Synthesis of MnTCF₃CH₂-3-PyP⁵⁺

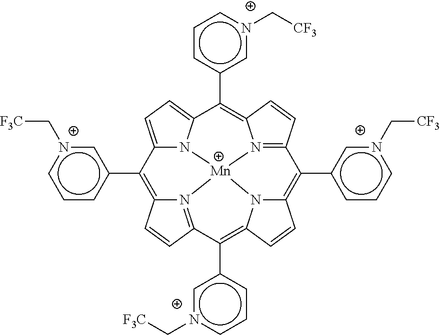

The fluoroalkylation of H₂T-3-PyP is carried out as in Example 1 except that an appropriate fluoroalkylating agent, such as (1,1-dihydroperfluoroalkyl)phenyliodonium triflates (Umemoto, T. and Gotoh, Y., Bull. Chem. Soc. Jpn. 1987, 60, 3307-3313; Umemoto, T. and Gotoh, Y., Bull. Chem. Soc. Jpn. 1991, 64, 2008-2010), is used instead of ethyl tosylate. The reaction between H₂T-3-PyP is carried out in anhydrous DMF at 50° C., protected from moisture. The progress of the reaction is monitored by TLC. The product is purified using a work-up procedure similar to that described in Example 1. Metallation of H₂TCF₃CH₂-3-PyP⁴⁺ with Mn²⁺ and purification to yield MnTCF₃CH₂-3-PyP⁵⁺ is accomplished as described in Example 1.

### Example 8

### Synthesis of MnTCF₃CH₂-2-PyP⁵⁺

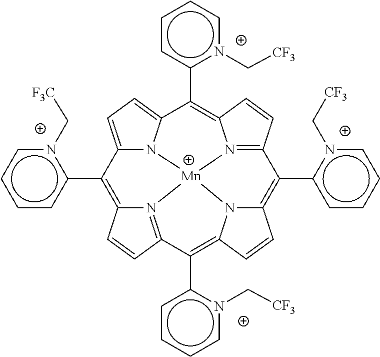

The fluoroalkylation of H₂T-2-PyP is carried out as in Example 7 except H₂T-2-PyP is used instead of H₂T-3-PyP. Metallation of H₂TCF₃CH₂-2-PyP⁴⁺ with Mn²⁺ and purification to yield MnTCF₃CH₂-2-PyP⁵⁺ is accomplished as described in Example 1.

### Example 9

### Protection of SOD-Deficient E. coli when Growing Aerobically by MnTnPr—OH-3-PyP

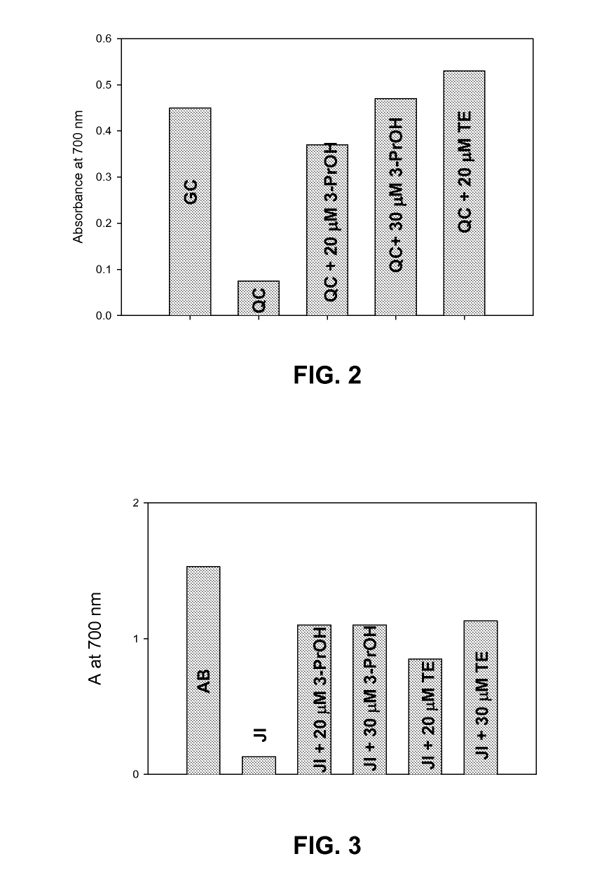

Escherichia coli (E. coli) strains used were AB1157, wild type (F-thr-1; leuB6; proA2; his-4; thi-1; argE2; lacY1; galK2; rpsL; supE44; ara-14; xyl-15; mtl-1; tsx-33), and JI1132, SOD-deficient, sodA-sodB-(same as AB1157 plus (sodA::mudPR13)25 (sodB-kan)1-Δ2). Both strains were obtained from J. A. Imlay. In addition to AB/JI strains another strain GC and its SOD-deficient analogue QC were used. The experiments were carried out as described in more detail in Reboucas et al., Dalton Trans., 2008, 1233-42. Briefly, cultures were grown aerobically in a 5 amino acid restricted medium (L-leucine, L-threonine, L-proline, L-arginine, L-histidine) in flasks on a water bath shaker at 37° C. and 200 rpm. The effect of the compound of Example 1 on the growth of these strains was followed turbidimetrically at 700 nm (to minimize the interference of compounds studied) and compared to the growth curves of both strains in the absence of Mn porphyrin (controls). Deionized water was used throughout the study. The effects are shown in FIG. 2 for one strain (QC and GC) and in FIG. 3 for other strain (AB and JI). FIG. 2 shows the aerobic growth of wild type (GC) and SOD-deficient strain (QC) of E. coli in minimal medium of 5 amino acids in the presence and absence of MnTE-2-PyP⁵⁺ and its alcohol analogue MnTnPrOH-3-PyP⁵⁺ at 19th hour, with MnTE-2-PyP⁵⁺ used as a positive control. FIG. 3 shows the aerobic growth of wild type (AB) and SOD-deficient strain (JI) of E. coli in minimal medium of 5 amino acids in the presence and absence of MnTE-2-PyP⁵⁺ and its alcohol analogue MnTnPrOH-3-PyP⁵⁺ at 19th hour, with MnTE-2-PyP⁵⁺ used as a positive control. At both 20 and 30 μM concentrations, MnTnPrOH-3-PyP was similar (FIG. 2) or better (FIG. 3) in protecting E. coli than MnTE-2-PyP⁵⁺. The effects varied with the type of the strain.

### Example 10

### Redox and Antioxidant Properties

Table indicating the redox properties (E_{1/2} of the Mn^{III}P/Mn^{II}P redox couple) and antioxidant (log k_{cat} for the dismutation of superoxide, O_{2′}⁻) properties of ortho and meta isomers of Mn N-substituted pyridylporphyrins. The E_{1/2} and log k_{cat} were determined as described in Reboucas et al., Dalton Trans., 2008, 1233-42. Rf is the ratio of the drug path over solvent path on thin-layer silica with water/acetonitrile/KNO₃ saturated water and is a measure of drug bioavailability. The data show that ortho and meta porphyrins all have high SOD activities, although ortho porphyrins are more SOD active. In addition, as the length of the pyridyl substituent increases the SOD activity of the ortho compounds drops while there is no change in the SOD activity of the meta compounds.

| Compound | Rf | E_{1/2}(mV) | logk_{cat} |
|---|---|---|---|
| MnTnPrOH-3-PyP | 0.05 | +52 | 6.83 |
| H2TnPrOH-3-PyP | 0.1 | | |
| MnTnPrOH-2-PyP | 0.05 | +238 | 7.38 |
| H2TnPrOH-2-PyP | 0.09 | | |

Rf is a measure of lipophilicity and is linearly related to log P where P is the partition coefficient between water and n-octanol. (Kos et al., Free Radic. Biol. Med., 47, 72-78, 2009, which is incorporated by reference herein).

### Example 11

### Protection Against Radiation Induced Cytotoxicity

### Cytotoxicity: XTT Cell Proliferation

XTT, a tetrazolium salt, is used to measure cell viability after irradiation exposure. XTT is reduced by viable mitochondria to formazan, which causes a colorimetric change that can be measured at 450 nm. Approximately 5×10⁴ cells are seeded per well in 96-well plates and incubated for 18 hours in fresh medium along with the compound at the final concentration indicated (3 replicates each). One set of plates is used as an unirradiated control and the other set was irradiated with 5 Gy. Five Gy is the optimal dose for observing efficient cytotoxicity within 48 hours. An XTT cell proliferation kit (Roche) is used to measure this cytotoxicity. All assays are performed in triplicate and the absorbance is read on a Spectramax plate reader. XTT is used to assess both the toxicity of the compounds and the effect of each compound on IR-induced cytotoxicity. All cells are pre-treated with the indicated compound two hours prior to being irradiated with 5 Gy and are assayed for viability 48 hours post-radiation. MnTnPrOH-3-PyP and MnTnPrOH-2-PyP reduce IR-induced cytotoxicity in WT cells in the amount of 50% as compared to untreated irradiated WT cells.

### Cytotoxicity: Annexin V/Propidium Iodide

Annexin V/propidium iodide staining is used as a second method to measure radiation-induced cytotoxicity. Cells are incubated in fresh medium along with the compounds at the final concentrations indicated. One set of samples is irradiated (5 Gy) to efficiently induce apoptosis within 48 hours. An annexin V/propidium iodide kit (BioVision) is used to detect apoptotic cells. The annexin V is labeled with fluorescein isothiocyanate (FITC) to detect phospholipid phosphatidylserine (PS) on the outer membrane of apoptotic cells; propidium iodide (PI) is used to detect necrotic cells. The assay is performed according to the manufacturer's instructions. Briefly, approximately 5×10⁵ cells are washed in PBS, resuspended in annexin V and PI staining solution for 15 min at room temperature and analyzed immediately by flow cytometry (Flow Cytometry Core, UCLA). Annexin V and propidium iodide staining is used to measure post-radiation apoptosis. 60% reduction in the percentage of apoptotic cells after radiation exposure is observed with MnTnPrOH-3-PyP and MnTnPrOH-2-PyP in wild type cells and 40% in A-T cells.

### DNA Damage: Gamma H2AX Immunofluorescence

Radiation-induced DNA damage recognition is measured by γ-H2AX immunofluorescence. Wildtype (WT) cells are propagated in fresh medium with the compounds at specified concentrations. Only WT cells were used for this assay since irradiation does not rapidly induces formation of γ-H2AX foci in an ATM-deficient cells pendent [465]. WT cells are collected at log phase of their growth cycle, after an 18-hour incubation with the indicated compounds. After treatment, the cells are irradiated with 2 Gy and assessed at 15 minutes. These conditions maximize the yield of DNA damaged cells while minimizing cytotoxicity. The cells are dropped onto cover slips and fixed with 4% paraformaldehyde, semi-permeabilized with 0.5% Triton-X 100 and washed with PBS. Cells are incubated with 1:400 dilution of mouse monoclonal antibody to γ-H2AX (Upstate Biotechnology), followed by 1:200 dilution of goat anti-mouse IgG antibody to immunoglobulin labeled with FITC (Jackson Immunochemicals) in PBS containing 10% FBS for 1.5 and 1 h, respectively. Slides are analyzed for γ-H2AX nuclear foci formation with FISH analysis software (Vysis) on a Leica DM RXA automated microscope equipped with Photometrix SenSyn. All slides are coded by one person and read blindly by another. MnTnPrOH-3-PyP and MnTnPrOH-2-PyP reduce IR-induced γ-H2AX immunofluorescent nuclear foci (IRIFs) by 57%.

### Example 12

### Carrageenan-Induced Pleurisy

Pleurisy is induced by carrageenan. Six to eight week old CD1 male mice are used. The mice are anesthetized with isoflurane and a skin incision is made at the level of the left sixth intercostal space. The underlying muscle is dissected and saline (0.2 ml) or saline containing 1% (w/v) λ-carrageenan (Sigma-Aldrich Ltd, 0.2 ml) is injected into the pleural cavity. The skin incision is closed with a suture and the animals are allowed to recover. At 4 h after the injection of carrageenan, the animals are killed. The chest is carefully opened and the pleural cavity is rinsed with 2 ml of saline solution containing heparin (5 U/ml) and indomethacin (10 μg/ml). The exudate and washing solution is removed by aspiration and the total volume is measured. Any exudate, which is contaminated with blood, is discarded. The amount of exudate is calculated by subtracting the volume injected (2 ml) from the total volume recovered. The leukocytes in the exudate are suspended in phosphate-buffer saline (PBS, 0.01M, pH7.4) and counted with an optical microscope in a Burker's chamber after vital Trypan Blue staining. MnTnPrOH-3-PyP and MnTnPrOH-2-PyP are able to decrease the volume of exudates, number of polymorphonuclear cells and myeloperoxidase activity (parameters of inflammation) to control levels at 0.3 mg/kg.

### Example 13

### Carrageenan-Induced Paw Oedema

Male Sprague-Dawley rats (300-350 g; Charles River, Milan) are used. They receive a subplantar injection of 0.1 mL saline containing 1% I-carrageenan in the right hindpaw. The inflammatory agent is given together with vehicle or in combination with MnTBAP (5, 25, and 50 mg/paw). The test agent is solubilised in saline solution and the injection volume is 0.1 mL. Control animals received the same volume of vehicle. The volume of the paw is measured by plethysmometry (model 7140; Ugo Basile) immediately after the injection. Subsequent readings of the volume of the same paw are carried out at 60-min intervals and compared to the initial readings. MnTnPrOH-3-PyP and MnTnPrOH-2-PyP are able to decrease the number of polymorphonuclear cells and myeloperoxidase activity (parameters of inflammation) to control levels at 0.3 mg/kg.

### Example 14

### Treatment with Hyperthermia and Mn Porphyrins

Treatment with hyperthermia and Mn porphyrins leads to a significant delay in tumor growth. In C57/BL6 mice, B16F10 melanoma cell line (1×106) is implanted in right flank. The mice are treated with hyperthermia (−41.5° C. for 1 hr) (1d, 5d, 8d) water bath and MnTnPrOH-3-PyP and MnTnPrOH-2-PyP (5 mg/kg twice per day). Tumor volumes are measured. Animals are sacrificed at day 9. Full delay of tumor growth is observed with the combination treatment of hyperthermia and Mn porphyrin.

### Example 15

### MnTnPrOH-2-PyP and MnTnPrOH-3-PyP Enhance Radiosensitivity of Tumor Vasculature In Vivo

4T1 window chamber tumors are randomized to treatment with PBS or MnTE-2-PyP⁵⁺, and radiation or sham-irradiation. A course of three fractions of radiation (5 Gy each, 12 hours apart) is followed immediately by daily administration of MnTnPrOH-2-PyP or MnTnPrOH-3-PyP (6 mg/kg/day) for three days. Tumors are imaged immediately after radiation (0 hours), and every day thereafter (24, 48, and 72 hours), and these images are used to calculate the tumor vascular length densities. Combined treatment results in significant tumor devascularization between 48 and 72 hours post-radiation.

### Example 16

### NCI-60 DTP Human Tumor Cell Line Screen

The effects of compounds according to Formula I on cancer cell lines are evaluated using the NCI-60 DTP Human Tumor Cell Line Screen. The NCI-60 DTP Human Tumor Cell Line Screen utilizes 60 different human tumor cell lines, representing leukemia, melanoma, and cancers of the lung, colon, brain, ovary, breast, prostate, and kidney. The screening is a two-stage process. First, a testing compound is evaluated against all 60 cell lines at a single dose of 10 μM. Second, a testing compound is evaluated against the 60 cell panel at five concentration levels.

The human tumor cell lines of the cancer screening panel are grown in RPMI 1640 medium containing 5% fetal bovine serum and 2 mM L-glutamine. For a typical screening experiment, cells are inoculated into 96 well microtiter plates in 100 μL at plating densities ranging from 5,000 to 40,000 cells/well depending on the doubling time of individual cell lines. After cell inoculation, the microtiter plates are incubated at 37° C., 5% CO₂, 95% air and 100% relative humidity for 24 h prior to addition of testing compound.

After 24 h, two plates of each cell line are fixed in situ with TCA, to represent a measurement of the cell population for each cell line at the time of drug addition (Tz). Testing compound is solubilized in dimethyl sulfoxide at 400-fold the desired final maximum test concentration and stored frozen prior to use. At the time of drug addition, an aliquot of frozen concentrate is thawed and diluted to twice the desired final maximum test concentration with complete medium containing 50 μg/mL gentamicin. Additional four, 10-fold, or ½ log serial dilutions are made to provide a total of five drug concentrations plus control. Aliquots of 100 μL of these different drug dilutions are added to the appropriate microtiter wells already containing 100 μL of medium, resulting in the required final drug concentrations.

Following addition of testing compound, the plates are incubated for an additional 48 h at 37° C., 5% CO₂, 95% air, and 100% relative humidity. For adherent cells, the assay is terminated by the addition of cold TCA. Cells are fixed in situ by the gentle addition of 50 μL of cold 50% (w/v) TCA (final concentration, 10% TCA) and incubated for 60 minutes at 4° C. The supernatant is discarded, and the plates are washed five times with tap water and air dried. Sulforhodamine B (SRB) solution (100 μL) at 0.4% (w/v) in 1% acetic acid is added to each well, and plates are incubated for 10 minutes at room temperature. After staining, unbound dye is removed by washing five times with 1% acetic acid, and the plates are air dried. Bound stain is subsequently solubilized with 10 mM trizma base, and the absorbance is read on an automated plate reader at a wavelength of 515 nm. For suspension cells, the methodology is the same except that the assay is terminated by fixing settled cells at the bottom of the wells by gently adding 50 μL of 80% TCA (final concentration, 16% TCA). Using the seven absorbance measurements [time zero, (Tz), control growth, (C), and test growth in the presence of drug at the five concentration levels (Ti)], the percentage growth is calculated at each of the drug concentrations levels. Percentage growth inhibition is calculated as:

[(Ti−Tz)/(C−Tz)]×100 for concentrations for which Ti>/=Tz

[(Ti−Tz)/Tz]×100 for concentrations for which Ti<Tz.

Three dose response parameters are calculated for each experimental agent: GI₅₀, TGI, and LC₅₀. Growth inhibition of 50% (GI₅₀) is calculated from [(Ti−Tz)/(C−Tz)]×100=50, which is the drug concentration resulting in a 50% reduction in the net protein increase (as measured by Sulphorhodamine-B (SRB) staining) in control cells during the drug incubation. The drug concentration resulting in total growth inhibition (TGI) is calculated from Ti=Tz. The LC₅₀ (concentration of drug resulting in a 50% reduction in the measured protein at the end of the drug treatment as compared to that at the beginning) indicating a net loss of cells following treatment is calculated from [(Ti−Tz)/Tz]×100=-50. Values are calculated for each of these three parameters if the level of activity is reached; however, if the effect is not reached or is exceeded, the value for that parameter is expressed as greater or less than the maximum or minimum concentration tested.

A single dose of testing compound reduces the growth of or is more than 50% lethal to at least one tumor tested, including leukemia, non-small cell lung cancer, colon cancer, CNS cancer, melanoma, ovarian cancer, renal cancer, prostate cancer, and breast cancer.

### Example 17

### Inhibition of Cancer Progression Using Compounds According to Formula I

### Animals

Female Fisher-344 rats, C57/B16 mice, and Balb/C mice are housed and treated in accordance with approved guidelines from the Duke University Institutional Animal Care and Use Committee.

### Tumors

4T1 tumors are grown in the flank of Balb/C mice by injecting a single cell suspension of tumor cells (10⁵). Tumor volumes are measured with calipers and calculated according to two diameters with the formula: v=(a²×b)/2, where v is the volume, a is the short diameter, and b is the long diameter. Animals are sacrificed once tumors reach five times their initial treatment volumes.

### Skinfold Window Chambers

Mice are anesthetized with sodium pentobarbital (80 mg/kg, i.p.), and a 1-cm diameter circular incision is made in the dorsal skin flap, over which a titanium chamber is surgically implanted. A 10-μL suspension of tumor cells (5×10³ cells) is then injected into the opposing flap of skin. A circular cover slip is placed over the incision, through which the vasculature, tissue, and tumor cells are visualized. Observations of window chamber tumors are performed daily on restrained, unanaesthetized mice with an inverted Zeiss fluorescence microscope (Carl Zeiss, Jena, Germany). Images are captured onto a personal computer with Scion Image software (Frederick, Md.) and analyzed with Adobe Photoshop (San Jose, Calif.). Tumor volume and vascular length densities are calculated as follows. Briefly, tumor volumes are calculated with the formula: d²(3.14/2), where the diameter is determined from low-power (2.5×) microscopy images by comparing pixel dimensions with micrometer values. Vascular length densities are measured from medium-power (5×) fields by using image analysis software (Scion Image) to trace the vascular network. Measurement of the sum length of all vessels within each tumor is then determined (in pixels) and converted to metric length by comparing pixel dimensions with micrometer values.

### Tumor Irradiation

Animals are randomized to treatment groups once flank tumors reach a mean volume of 200 mm³ (n=5 per group), and window chamber tumors are 1 mm in diameter (n=5 per group). Tumor-bearing Balb/C mice are restrained, unanaesthetized, in a plastic tube and placed in a Mark IV Cesium irradiator (dose rate=7 Gy/min). The mice are positioned behind a lead shield allowing only the tumor-bearing area to remain in the treatment field. Three doses of 5 Gy are administered, separated by 12 h each.

### Drug Treatment

Compounds according to the invention are prepared in sterile phosphate buffered saline (PBS) and are administered by i.p. injections (6 mg/kg and 100 mg/kg, respectively) according to the schedules.

### Statistics

Unless otherwise noted, data are reported as mean±standard deviation. Statistical significance is determined with a Student t test or analysis of variance, where appropriate, and p values less than 0.05 are considered significant.

### Example 18

### Potentiation of Cells for Anti-Cancer Treatment

The Dose Enhancing Factor (DEF) is a ratio of the enhancement of cell growth inhibition elicited by the test compound in the presence of bleomycin compared to bleomycin alone. The test compounds are used at a fixed concentration of 25 μM. Bleomycin is used at a concentration of 0.5 μg/mL. The DEF is calculated from the formula:

$$\frac{{Growth}_{TC}}{{Growth}_{Control}} \times \frac{{Growth}_{bleo}}{{Growth}_{( bleo + TC )}}$$

where Growth_{TC} is cell growth in presence of the test compound;

Growth_{Control} is cell growth of control cells;

Growth_{bleo} is cell growth in presence of bleomycin; and

Growth_{(bleo+TC)} is cell growth in presence of bleomycin and the test compound.

Cell growth is assessed using the sulforhodamine B (SRB) assay (Skehan, P., et al., 1990, J. Natl. Cancer Inst., 82, 1107-1112). 2,000 HeLa cells are seeded into each well of a flat-bottomed 96-well microtiter plate in a volume of 100 μL and incubated for 6 hours at 37° C. Cells are either replaced with media alone or with media containing the test compound at a final concentration of 25 μM. Cells are allowed to grow for a further 1 hour before the addition of bleomycin to either untreated cells or test compound treated cells. Cells untreated with either bleomycin or test compound are used as a control. Cells treated with test compound alone are used to assess the growth inhibition by the test compound.

Cells are left for a further 16 hours before replacing the media and allowing the cells to grow for a further 72 hours at 37° C. The media is then removed and the cells fixed with 100 μL of ice cold 10% (w/v) trichloracetic acid. The plates are incubated at 4° C. for 20 minutes and then are washed four times with water. Each well of cells is then stained with 100 μL of 0.4% (w/v) SRB in 1% acetic acid for 20 minutes before washing four times with 1% acetic acid. Plates are then dried for 2 hours at room temperature. The dye from the stained cells is solubilized by the addition of 100 μL of 10 mM Tris Base into each well. Plates are gently shaken and left at room temperature for 30 minutes before measuring the optical density at 564 nM on a Microquant microtiter plate reader.

1. A compound according to Formula I: 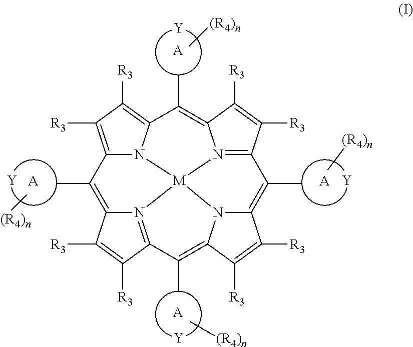
wherein each A is independently selected from the group consisting of an unsubstituted or substituted heteroaryl group and aryl group;
wherein each Y is independently selected from the group consisting of a CH and a heteroatom;
wherein each R₄ is independently —R₁—X—R₂;
wherein each R₁ is independently an unsubstituted or substituted alkylene;
wherein each X is independently selected from the group consisting of a direct bond and a heteroatom;
wherein each R₂ and R₃ are independently selected from the group consisting of hydrogen, halogen, hydroxyl, alkoxy, alkyl, alkenyl, alkynyl, cycloalkyl, heterocycloalkyl, amino, amide, nitro, keto, oxo, carboxylic acid, carboxyl, aryl, heteroaryl, thiol, thioalkyl, thioester, disulfide, phosphine, carbonyl, carbonylamino, formyl, sulfonyl, sulfonylamino, cyano, isocyano, C₁₋₄ alkyl aryl, and C₁₋₄ alkyl heteroaryl;
wherein each n is independently 0 to 2;
wherein M is selected from the group consisting of Mn, Fe, Co, Ni, Cu, V, and 2 hydrogens;
wherein at least one —R₁—X—R₂ contains at least one heteroatom; and
wherein at least one Y is N—R₄.

2. A compound according to claim 1, wherein M is Mn.

3. A compound according to claim 1, wherein at least one A is heteroaryl.

4. A compound according to claim 3, wherein the at least one A is substituted pyridyl.

5. A compound according to claim 1, wherein R₁ is an alkylene.

6. A compound according to claim 5, wherein the alkylene has from 4 to 10 carbon atoms.

7. A compound according to claim 6, wherein the alkylene has 6 carbon atoms.

8. A compound according to claim 1, wherein X is O.

9. A compound according to claim 1, wherein R₂ is alkoxy.

10. A compound according to claim 9, wherein the alkoxy comprises a trifluoromethyl group.

11.-12. (canceled)

13. The compound according to claim 1, wherein at least one R₃ is H.

14. The compound according to claim 1, wherein at least one R₄ is alkyl substituted with hydroxyl or carboxylic acid.

15. The compound according to claim 1, wherein at least one R₄ comprises an ester group.

16.-18. (canceled)

19. A compound according to claim 1, wherein the compound is selected from the group consisting of:

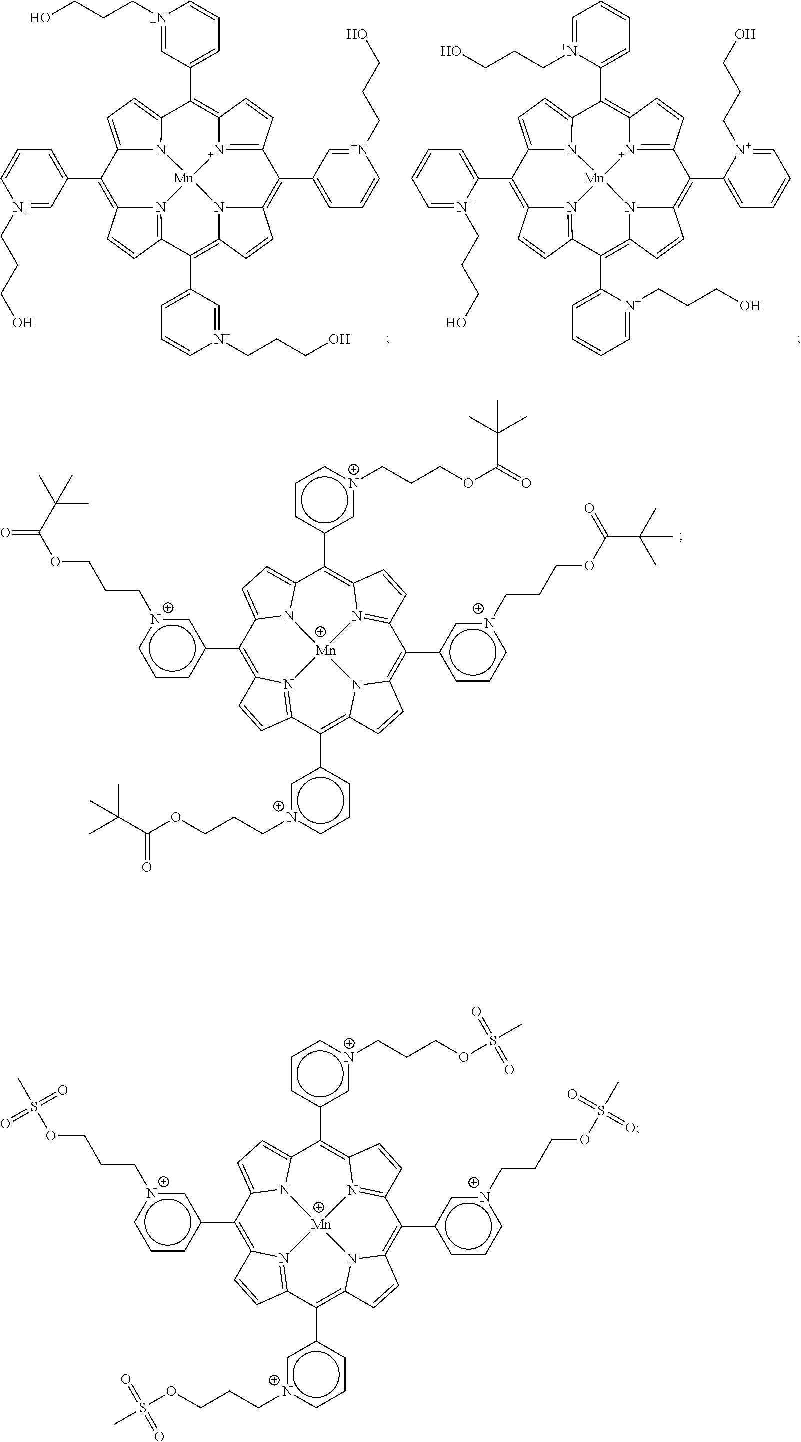

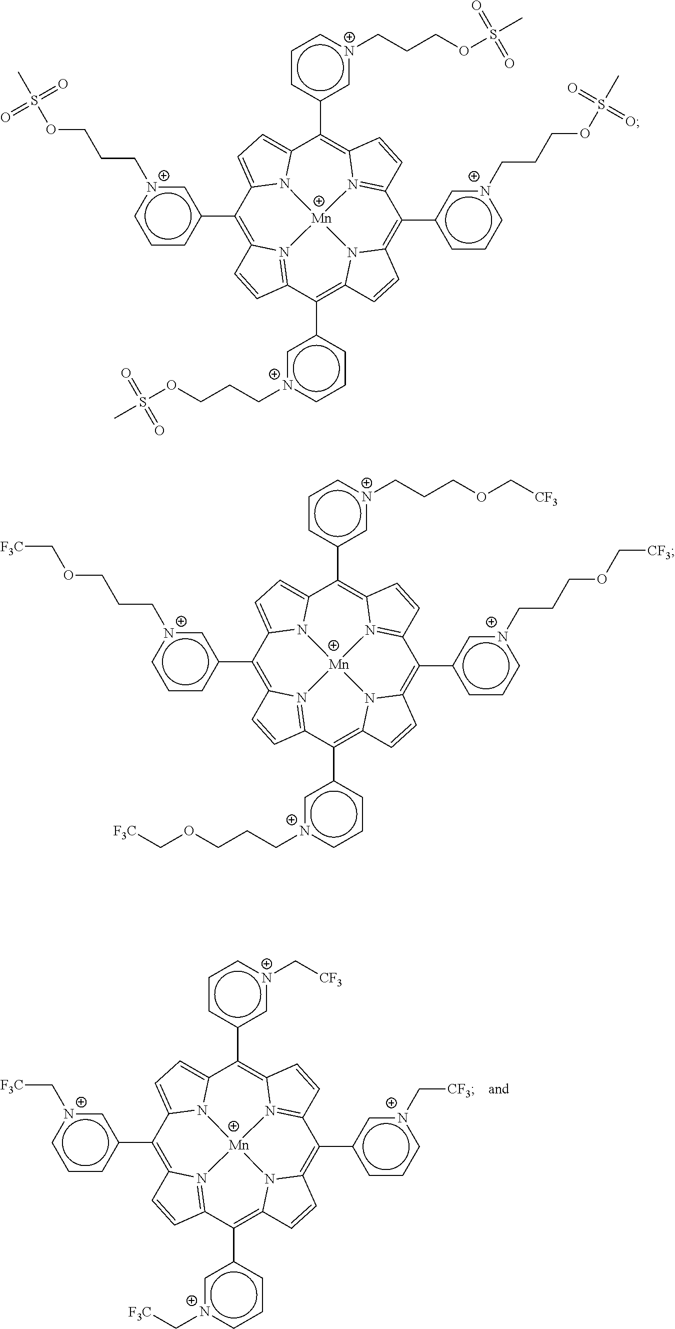

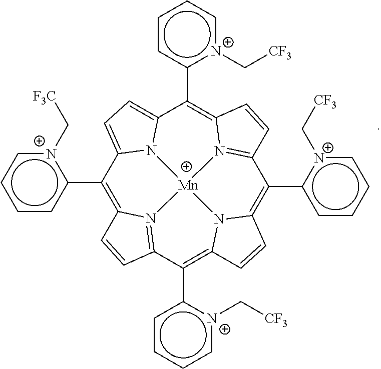

20. A method of reducing oxidative stress in a cell, the method comprising contacting the cell with an effective amount of a compound according to claim 1.

21. A method of treating a disease or disorder, the method comprising administering to a patient in need thereof an effective amount of a compound according to claim 1, wherein the disease or disorder is selected from the group consisting of central nervous system injuries, stroke, spinal cord injury, cancer, ischemia/reperfusion injuries, cardiovascular injuries, arthritis, sickle cell disease, radiation injury, auto-immune diseases, diabetes, morphine tolerance, drug dependence/addiction and inflammatory conditions.

22. The method of claim 21, wherein the central nervous system injury is selected from the group consisting of ALS, Alzheimer's, multiple sclerosis, and Parkinson's.

23. The method of claim 21, wherein the compound is administered with an active agent.

24.-25. (canceled)

26. A pharmaceutical composition comprising a pharmaceutically acceptable carrier and a compound according to claim 1.

27. A method of treating a subject with a disorder associated with oxidative stress, the method comprising administering to the subject an effective amount of a compound according to claim 1.

28.-29. (canceled)
